(19) United States
(12) Patent Application Publication
(10) Pub. No.: US 2022/0076460 A1
(43) Pub. Date: Mar. 10, 2022

(54) SYSTEM, METHOD AND COMPUTER-ACCESSIBLE MEDIUM FOR IMAGE RECONSTRUCTION OF NON-CARTESIAN MAGNETIC RESONANCE IMAGING INFORMATION USING DEEP LEARNING

(71) Applicants: JOHN THOMAS VAUGHAN, JR., New York, NY (US); SAIRAM GEETHANATH, New York, NY (US); PEIDONG HE, New York, NY (US)

(72) Inventors: JOHN THOMAS VAUGHAN, JR., New York, NY (US); SAIRAM GEETHANATH, New York, NY (US); PEIDONG HE, New York, NY (US)

(73) Assignee: The Trustees of Columbia University in the City of New York, New York, NY (US)

(21) Appl. No.: 17/475,630

(22) Filed: Sep. 15, 2021

Related U.S. Application Data
(63) Continuation of application No. PCT/US2020/022980, filed on Mar. 16, 2020.
(60) Provisional application No. 62/819,125, filed on Mar. 15, 2019.

(51) Int. Cl. G06T 11/00; G16H 30/40; G06N 3/04

(52) U.S. Cl. CPC G06T 11/005; G06N 3/04; G16H 30/40

(57) ABSTRACT

An exemplary system, method, and computer-accessible medium for generating a Cartesian equivalent image(s) of a portion(s) of a patient(s), can include, for example, receiving non-Cartesian sample information based on a magnetic resonance imaging (MRI) procedure of the portion(s) of the patient(s). and automatically generating the Cartesian equivalent image(s) from the non-Cartesian sample information using a deep learning procedure(s). The non-Cartesian sample information can be Fourier domain information. The non-Cartesian sample information can be undersampled non-Cartesian sample information. The MRI procedure can include an ultra-short echo time (UTE) pulse sequence The UTE pulse sequence can include a delay(s) and a spoiling gradient. The Cartesian equivalent image(s) can be generated by reconstructing the Cartesian equivalent image(s). The Cartesian equivalent image(s) can be reconstructed using a sampling density compensation with a tapering of over a particular percentage of a radius of a k-space, where the particular percentage can be about 50%.

### CROSS-REFERENCE TO RELATED APPLICATION(S)

This application relates to and claims priority from U.S. Patent Application No. 62/819,125. filed on Mar. 15, 2019, the entire disclosure of which is incorporated herein by reference.

### FIELD OF THE DISCLOSURE

The present disclosure relates generally to magnetic resonance imaging (“MRI”), and more specifically, to exemplary embodiments of exemplary system, method and computer-accessible medium for image reconstruction of non-Cartesian magnetic resonance imaging information using deep learning.

### BACKGROUND INFORMATION

Automated transform by manifold approximation (“AUTOMAP”) describes a network that contains three fully connected network layers and three fully convolutional network layers. (See, e.g., Reference 7). The drawback of the fully connected network is that it requires a considerable amount of memory to store all the variables, especially when the resolution of the image is large. Additionally, the system docs not contain original phase information of the k-space. Instead, such system uses the synthetic phase to the k-space, and facilitates the conversion of any images from image-net to their training examples. Other methods focused more on pre-processing before Fourier transform (see. e.g., Reference 8) or post-processing after the Fourier transform. (See, e.g., Reference 9). These include decoration of k-space using deep learning, or removal of artifact after Fourier transform.

Thus, it may be beneficial to provide an exemplary system, method and computer-accessible medium for image reconstruction of non-Cartesian MRI information using deep learning which can overcome at least some of the deficiencies described herein above.

### SUMMARY OF EXEMPLARY EMBODIMENTS

An exemplary system, method, and computer-accessible medium for generating a Cartesian equivalent image(s) of a portion(s) of a patient(s), can include, for example, receiving non-Cartesian sample information based on a magnetic resonance imaging (MRI) procedure of the portion(s) of the patient(s), and automatically generating the Cartesian equivalent image(s) from the non-Cartesian sample information using a deep learning procedure(s). The non-Cartesian sample information can be Fourier domain information. The non-Cartesian sample information can be undersampled non-Cartesian sample information. The MRI procedure can include an ultra-short echo time (UTE) pulse sequence. The UTE pulse sequence can include a delay(s) and a spoiling gradient. The Cartesian equivalent image(s) can be generated by reconstructing the Cartesian equivalent image(s). The Cartesian equivalent image(s) can be reconstructed using a sampling density compensation with a tapering of over a particular percentage of a radius of a k-space, where the particular percentage can be about 50%.

In some exemplary embodiments of the present disclosure, the Cartesian equivalent image(s) can be reconstructed by gridding the non-Cartesian sample information to a particular matrix size. The Cartesian equivalent image(s) can be reconstructed by performing a 3D Fourier transform on the non-Cartesian sample information to obtain a signal intensity image(s). The deep learning procedure(s) can include at least 20 layers. The deep learning procedure(s) can include convolving an input at least twice. The deep learning procedure(s) can include max pooling the second layer. The deep learning procedure(s) can include convolving or max pooling a first 10 layers. The deep learning procedure(s) can include forming a 13^{th} layer by concatenating a 9^{th} layer with a 12^{th} layer. The deep learning procedure(s) can include convolving a last 4 layers. The deep learning procedure(s) can include maintaining a particular resolution from layer 13 to layer 18. The deep learning procedure(s) can include 13 convolutions, 4 deconvolutions, and 4 combinations of maxpooling and convolution.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary diagram illustrating code used for image reconstruction according to an exemplary embodiment of the present disclosure;

FIG. 2 is an exemplary network sketch map according to an exemplary embodiment of the present disclosure;

FIG. 3 is a set of exemplary reconstructed images according to an exemplary embodiment of the present disclosure;

FIG. 4 is a set of exemplary images of radial reconstruction according to an exemplary embodiment of the present disclosure;

FIG. 5A is an exemplary random phase map according to an exemplary embodiment of the present disclosure;

FIG. 5B is an exemplary image of actual slices from an American College of Radiology phantom according to an exemplary embodiment of the present disclosure:

FIG. 5C is an exemplary image of the actual slices from FIG. 5B overlayed using a random phase map according to an exemplary embodiment of the present disclosure;

FIG. 5D is an exemplary image of actual slices from an Alzheimer's Disease Neuroimaging Initiative phantom according to an exemplary embodiment of the present disclosure;

FIG. 5E is an exemplary image of the actual slices from FIG. 5D overlayed using a random phase map according to an exemplary embodiment of the present disclosure;

FIGS. 5F and 5H are exemplary phase angle illustrations according to an exemplary embodiment of the present disclosure;

FIGS. 5G and 5I are exemplary phase angle illustrations having a random phase map applied thereto according to an exemplary embodiment of the present disclosure:

FIG. 6 is an exemplary image, and associated slices in an axial plane, of an orthogonal slice of an American College of Radiology phantom according to an exemplary embodiment of the present disclosure;

FIG. 7 is an exemplary image and corresponding slice, of an Alzheimer's Disease Neuroimaging Initiative phantom according to an exemplary embodiment of lire present disclosure;

FIG. 8A is a set of exemplary images of training data samples of an American College of Radiology phantom slice and an Alzheimer's Disease Neuroimaging Initiative phantom slice according to an exemplary embodiment of the present disclosure;

FIG. 8B is a training graph of the training data samples shown in FIG. 8A according to an exemplary embodiment of the present disclosure;

FIG. 9 is a set of exemplary image reconstructions of accelerated radial imaging according to an exemplary embodiment of the present disclosure;

FIG. 10 is a set of images having different noise levels according to an exemplary embodiment of the present disclosure;

FIG. 11 is an exemplary table comparing various datasets according to an exemplary embodiment of the present disclosure;

FIG. 12 is a flow diagram of an exemplary method for generating a Cartesian equivalent image of a patient according to an exemplary embodiment of the present disclosure; and

FIG. 13 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and appended claims.

### DETAILED DESCRIPTION OF EXEMPLARY EMBODIMENTS

Ultra-short echo time (“UTE”) sequences (see. e.g., Reference 10) utilize rapid switching between transmit and receive coils, which can be challenging to implement without a deep understanding of vendors specific pulse programming environments. Pulseq is an open source tool and file standard capable of programming multiple vendor environments and multiple hardware platforms. The exemplary Pulseq can be used to simplify and facilitate rapid prototyping of such sequences. ImRiD is a carrier of mathematical transform from frequency domain 10 space domain. ImRiD can contain all the information of k-space including the phase and magnitude of the phantom. Various exemplary deep learning image reconstruction models can use the dataset for training.

The exemplary deep learning based image reconstruction procedure can learn the mathematical transform from the k-space directly to the image space for non-Cartesian k-space sampling. The Cartesian Fourier transform is already robust and fast. Therefore, there is no need to replace that by deep learning. For Cartesian space, deep learning can have a superior performance in removing trajectory-related artifacts, and can outperform traditional mathematical transforms in sub-sample scenarios. To train the exemplary network, a ground truth and corresponding input can be used. In this ease, the input can be subsampled k-space, and the ground that the neural network can match can be the image reconstructed from the full k-space.

### Exemplary Method

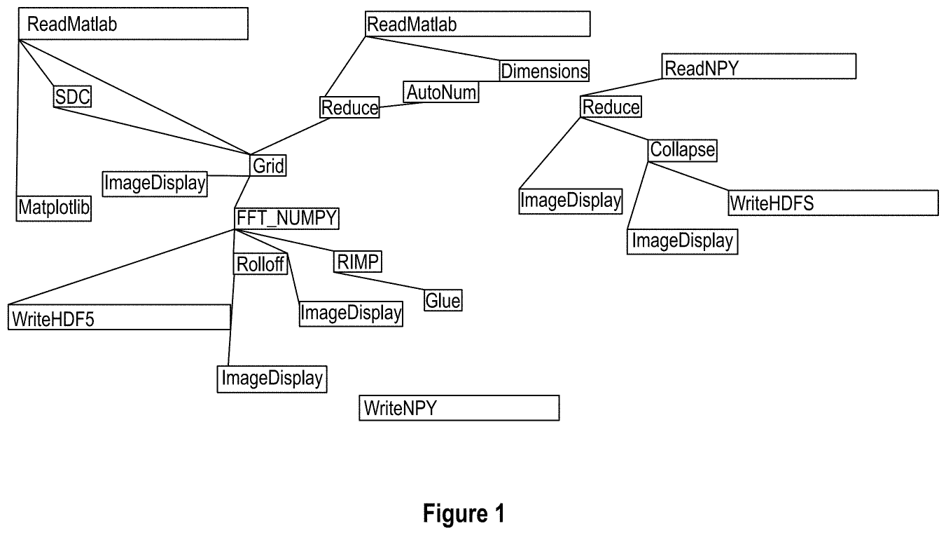

Pulseq based code was prepared for the 3D radial UTE sequence to generate sequence related files and k-space trajectory. In Pulseq, temporal behaviors in the scanner can be defined as a block. In each block, several events can be explicitly defined based on system constraints and specific absorption rate (“SAR”). In the exemplary code, after the repetition time (“TR”). the echo time (“TE”). the field of view (“FOV”), slew rate, maximum gradient, and radio frequency (“RF”) ring-down time, were determined, a for loop was constructed, in each iteration, and one spoke was specified. For the UTE sequence, it contains a short delay to satisfy the RF ring-down time; gradients Gx, Gy, Gz, and analog to digital conversion (“ADC”) activated for readout; another short delay and spoiling gradient. The last component of the Pulseq code can be generating the sequence file for the scanner to execute, and trajectory for later reconstruction task. The reconstruction included sampling density compensation with tapering over 50% of the radius of the k-space. The reconstruction was gridded to a matrix size of 256×256×256, followed by a 3D Fourier Transform to obtain signal intensity images. FIG. 1 shows an exemplary diagram illustrating code used for image reconstruction according to an exemplary embodiment of the present disclosure.

In particular, FIG. 1 illustrates the programming plot of the graphical programming interface (“GPI”) for reconstruction. The graphics code can be used by the exemplary system, method and computer-accessible medium to load the k-space trajectory and the acquired data in MATLAB formal, perform a Fourier transform for each channel, and display images in each channel and all channels combined. FIG. 1 describes the workflow of reconstruction of non-Cartesian k-space data given the trajectory, which is illustrated using an open source software Graphical Programming Interface. The workflow includes components to compensate for sampling density, grid the data on to a Cartesian grid and Fourier Transform to obtain the exemplary image.

### Exemplary Imaging

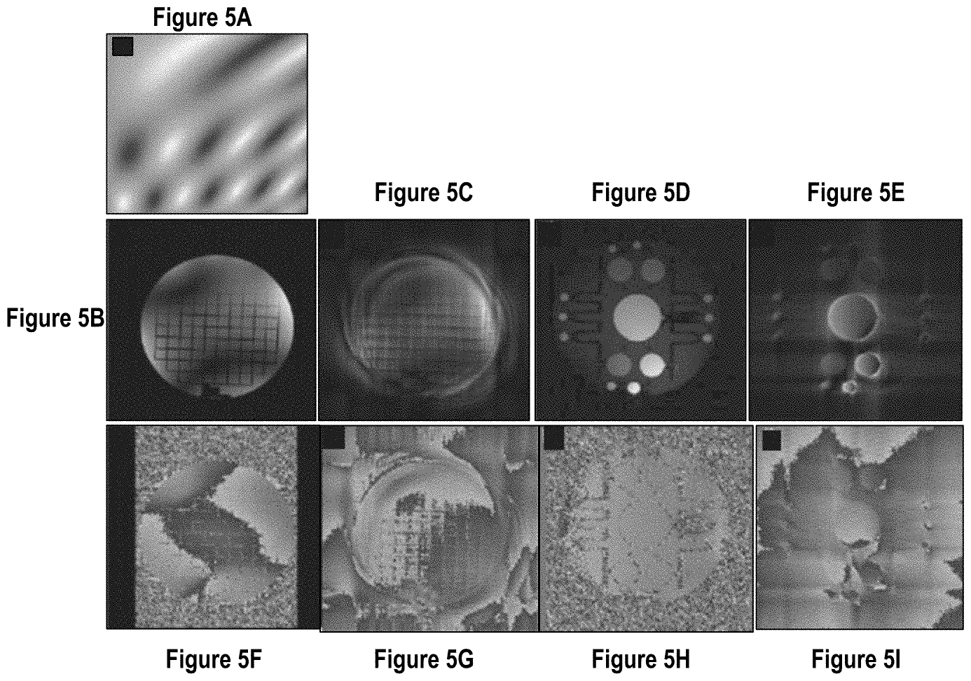

A 3D T1 weighted MP-RAGE (see. e.g., Reference 11) scan of the American College of Radiology (“ACR”) phantom (see. e.g., Reference 12) was acquired on a 3T Siemens Prisma scanner. The acquisition parameters were: FOV=256×256×192 mm³, TI=900 ms. flip angle=8°, TR=2300 ms, isotropic resolution of 1.05 mm with a matrix size of 255×255×192. The unfiltered k-space was Fourier transformed to provide a 3D complex magnetic resonance (“MR”) image volume. Similar data from the Alzheimer's disease Neuroimaging Initiative (“ADNI”) phantom (see. e.g., Reference 13) was also acquired with an identical protocol. This was performed utilizing T1 targets available in phantoms for quantitative imaging (e.g., or direct reconstruction methods). Orthogonal slices were extracted for the purpose of training and validation. In addition, arbitrary slices were chosen by indicating the vector normal to the desired plane. Then the corresponding k-space mapping was obtained by performing the inverse Fourier transform. The MATLAB code to leverage these planes was used to generate a particular number of arbitrary slices provided in the GitHub repository. (See. e.g., Reference 14). To illustrate the benefits of phase in MR reconstructions, the k-space resulting from the magnitude of the obtained complex images was synthesized using the Fourier transform. These synthetic k-spaces were then multiplied with exemplary random phase maps as showed in FIGS. 5A-5I. In particular, FIG. 5A illustrates an exemplary random phase map. FIG. 5B shows an exemplary image of actual slices from an ACR phantom. FIG. 5C illustrates an exemplary image of the actual slices from FIG. 5B overlayed using a random phase map, FIG. 5D shows an exemplary image of actual slices from an ADNI phantom, FIG. 5E illustrates an exemplary image of the actual slices from FIG. 5D overlayed using a random phase map, FIGS. 5F and 5H show exemplary phase angle illustrations, and FIGS. 5G and 5I show exemplary phase angle illustrations having a random phase map applied thereto. These maps were generated based on a random combination of sinusoids using MATLAB (The Mathworks Inc. MA). The magnitude and phase images resulting from the original and synthesized k-space were computed. For an exemplary training process, the full k-space information of an image can be sub-sampled by any suitable k-space sampling methods (e.g., radial, spiral). The corresponding actual slice image can then be the ground truth that the resampled k-space can be trained against.

### Exemplary Deep Learning Image Reconstruction

For the training process, 2D image slice was obtained from the raw data and reshaped to an image size of 256×256. The slicing from 3D volume can either be orthogonal or arbitrary. Orthogonal slicing was performed along the third dimension. In arbitrary slicing, to ensure the resolution to be identical when slicing docs not obtain enough pixels to fulfill the resolution, a noise map was generate based on the noise of the no signal area of the data, and randomly assigned to the empty region to form the slice with identical an resolution of 256×256. Sub-sampled k-space data (e.g., radial k-space sampling) was also obtained by using the Michigan Image Reconstruction Toolbox (“MIRT”) (see, e.g., Reference 15) from a raw image(s) with real and imaginary information. The sub-sampled radial k-space was then inverse non-uniform fast Fourier transformed (“NUFFT”) to radial reconstructed images. 2D FFT was performed to transform radial reconstructed images to 256×256 k-space, which has the same resolution as the ground truth slice.

The input for each data point to two 256×256 k-space vectors was separated, one for real pan and one for imaginary part, and normalized by log function then sealed to 0 to 100. The input was then reshaped to a long vector which has the length of 131072(65536 for real part and the rest 65536 for imaginary part). The training label was the absolute value of the ground truth slice, also sealed to 0 to 100. Normalization formula for k-space data included, for example:

$$\begin{matrix}x = log ( x + 1 ) & [ 1 ] \\ x = \frac{x - min ( x )}{max ( x ) - min ( x )} * 100 & [ 2 ]\end{matrix}$$

The label was the absolute value of the corresponding ground truth image and also being normalized by the formula 2 to 0 to 100. The exemplary U-net model utilized was based on Python programming language, and TensorFlow, Numpy, and Scipy packages were used to construct the model. The training examples were 7680 k-space data and corresponding images. The training process had 300 epochs and the batch size was 16. Adam optimizer and loss functions were utilized to the reduce mean of square loss between the output and the ground truth. The 0.5 on the left shown in the exemplary formula below can be to offset the 2 when performing a derivative.

loss=0.5(Y_{predict}−Yₜᵣᵤₑ)² [3]

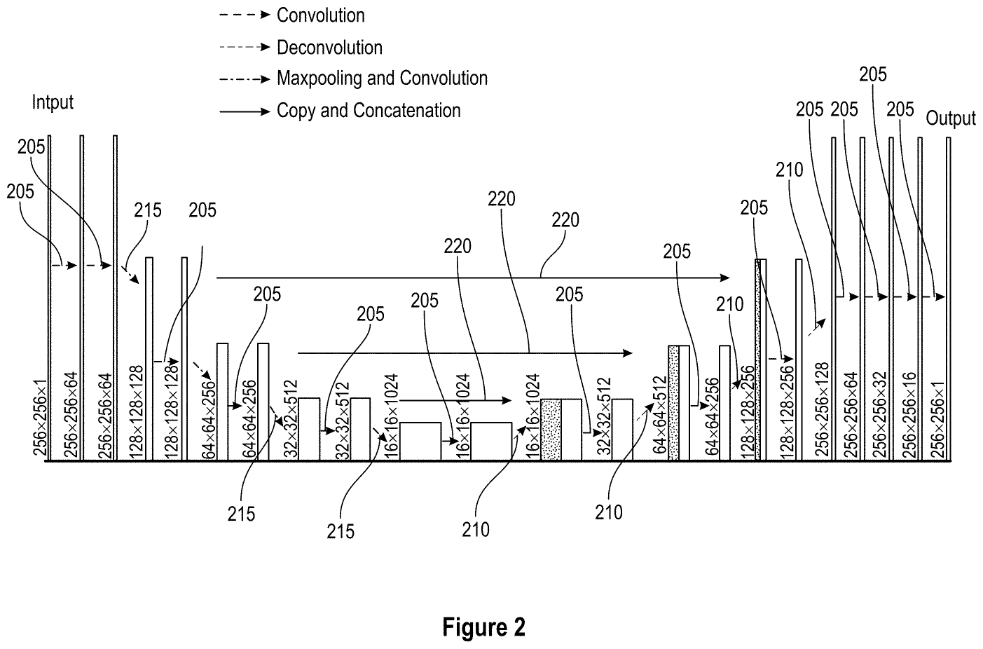

The input k-space vector can be the 2D Fourier transform result of the image that formed by an inverse NUFFT of radial k-space sub-sampled from full k-space. The full k-space can be Fourier transformed from a complex image slice. The exemplary U-net network implemented contained 19 convolution layers, 4 max pooling layers, and 5 deconvolution layers. FIG. 2 shows an exemplary network sketch map according to an exemplary embodiment of the present disclosure. The resolution of each layer is indicated at the bottom of the layer of FIG. 2. Arrows 205 in such diagram indicate convolution, arrows 210 indicate deconvolution, arrow's 215 indicate max pooling and then convolution, and arrows 220 indicate copy and then concatenation.

As shown in FIG. 2, the input was convolved two times and max pooling was performed before the next layer. The max pooling operation can also be followed by increasing the density of the layer. Convolution and max pooling were repeated until the 10th layer. From the 12th layer, the deconvolution was performed and the next layer concatenated with the 9th layer to form the 13th layer. The 13th layer was used for convolution and deconvolution. The same operation was repeated until layer 18 where the same resolution was maintained and 4 convolutions were performed to generate the exemplary result. In interpolation, which is shown by arrows 215, the max pooling can be separate layer variables or a function in convolutional operation. The result of deconvolutions can also be a separate layer or a function in the next layer.

The exemplary model was built in Python in TensorFlow framework. The activation function used can be rectified linear unit (“ReLu”). and the kernel size can be: 5×5 except the last layer can have a kernel size of 3×3. The training was performed on a machine with 4 Nvidia 1080 Ti graphics cards, 128 GB of RAM and an Intel i9-7980XE CPU.

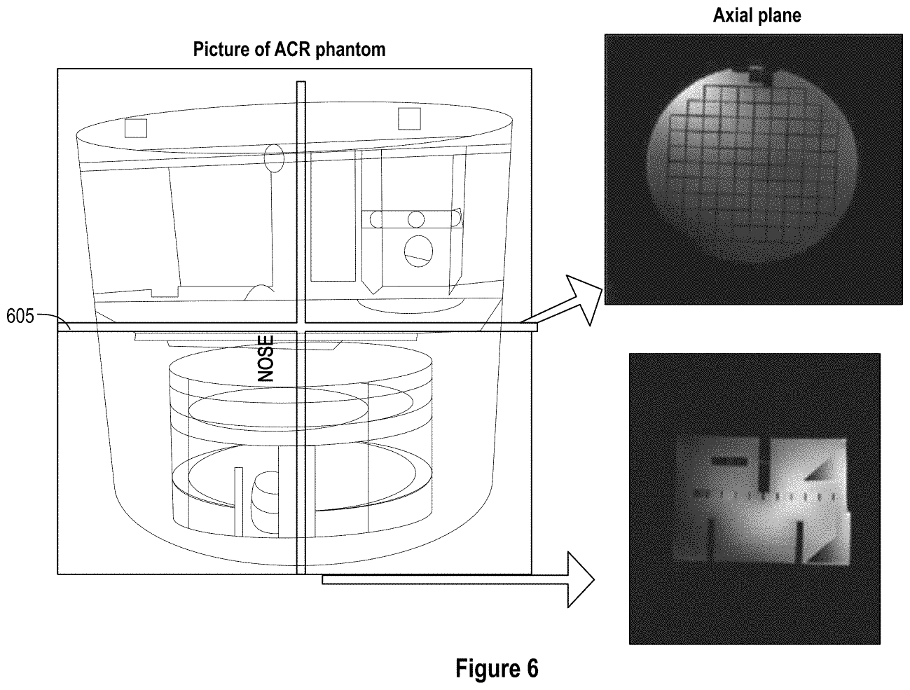

ImRiD was selected for the exemplary training dataset. It includes fully sampled scan data for ADNI and ACR phantoms. FIG. 6 shows an exemplary image, and associated slices in an axial plane, of an orthogonal slice of an ACR according to an exemplary embodiment of the present disclosure. The position of the slice is visualized by line 605 in the phantom picture.

These images were acquired with a resolution of 0.7 mm isotropic with a matrix size of 255×255×192, TI=900 ms, flip angle=8°, TR=2300 ms, 3D MP-RAGE sequence was applied to ACR and ADNI phantom to obtain the ground truth volume. These images were resized to 256×256×192 without loss of phase information. The training examples were 7680 k-space data and corresponding images. The training process had 300 epochs and the batch size was 16. An Adam optimizer was used and the loss function was the reduced mean of square loss between the output and the cost function. Each epoch took about 500 seconds to complete.

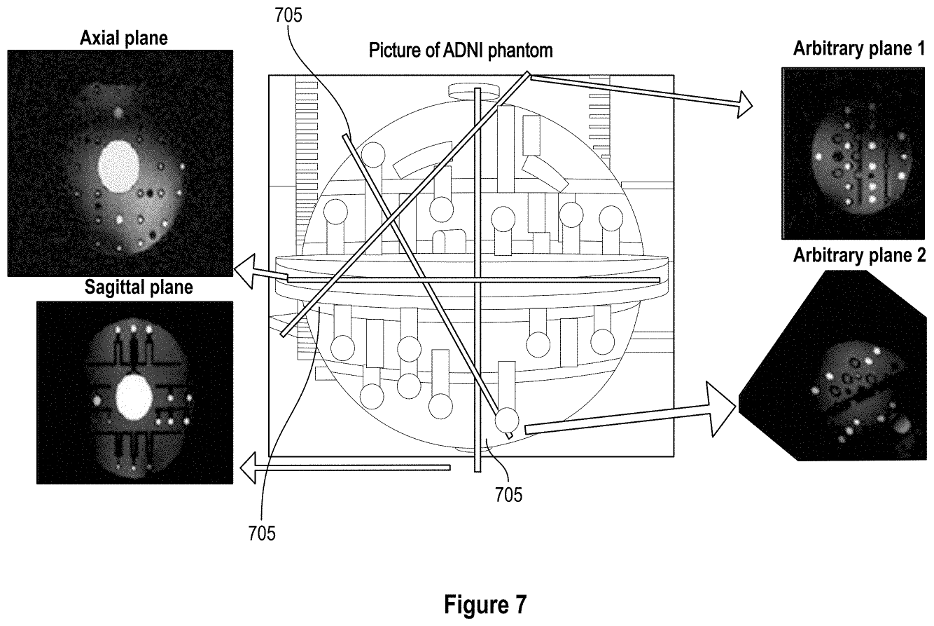

FIG. 7 shows the image of the ADNI phantom and the arbitrary planes and sagittal, axial plane selected for slicing according to an exemplary embodiment of the present disclosure. Orthogonal slices or arbitrary slices (e.g., represented by lines 705) can be specified and extracted from 3D fully sampled volume by indicating the vector normal to the desired plane. For the exemplary noise experiment, random noise at different noise levels (e.g., 0.01, 0.05, 0.1, 0.2) were added to the image (e.g., in the real and imaginary parts) that later transformed to k-space for training. This image was then used to generate Cartesian k-space and normalized for testing.

For the exemplary under-sample k-space experiment, the full k-space was retrospectively sub-sampled by skipping the spokes in radial k-space by 50% and 75%. Then the sub-sampled radial reconstructed image was generated and then Fourier transformed to k-space for the testing input. All normalization was done at the same scale for both the training data set and the testing data set.

### Exemplary Results

The Corresponding UTE sequence was generated and played in a SIEMENS Prisma scanner. A ADNI phantom and one subject sequence was demonstrated on a Siemens 3T Prisma with body coil on the ADNI phantom and knee imaging of a healthy volunteer (e.g., as part of IRB approved study); TR/TE=20/0.2 ms; 51472 spokes; 256×256×128 mm³; and the data was reconstructed offline using a GPI. The in vitro data illustrated the ability of the exemplary sequence to depict contrast and resolution contained in the ADNI phantom. The in vivo images of the knee yielded visualizations of the medial collateral ligament and synovial fluid in the sagittal views. For the reconstruction, the Krad and Taper variables in sampling density correction (“SDC”) were modified to determine the best value for reconstruction. A Taper value of 0.9 and Krad value of 0.8 were chosen for superior reconstruction results.

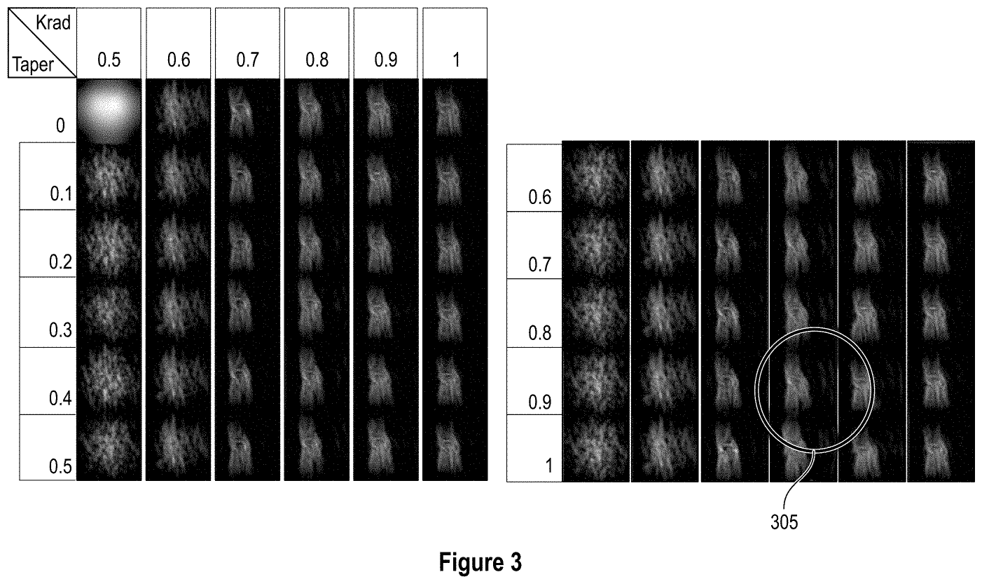

FIG. 3 shows a set of exemplary reconstructed images according to an exemplary embodiment of the present disclosure. In particular, FIG. 3 illustrates the effect of the radius and the taper in the sampling density correction on the image quality. Element 305 shown therein depicts the chosen image based on image quality.

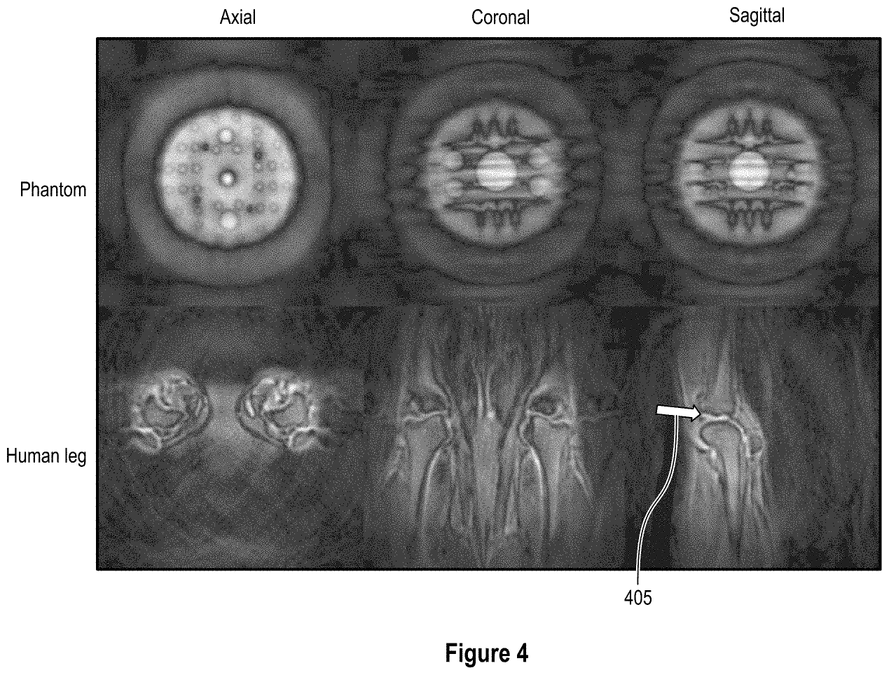

FIG. 4 shows a set of exemplary images of radial reconstructions according to an exemplary embodiment of the present disclosure. In particular, FIG. 4 illustrates the axial, coronal and sagittal image of the ADNI phantom and legs of the subject. Arrow 405 in FIG. 4 indicates the cartilage. The top three images show the axial, coronal and sagittal plane of the ADNI phantom. The lower three images show the axial, coronal and sagittal plane of the subject's knee in the image. The cartilage tissue between the femur and tibia is visible. The image was extracted from the 3D volume. The result was in 3D because the UTE sequence was sampled in 3D.

The body coil switching time can dictate the UTE that can be achieved. The exemplary implementation can be flexible to accommodate other hardware specifications as well. The exemplary demonstration is shown on a body coil. The coil closer to the knee can enhance signal-to-noise ratio. Coil selection may not impact the exemplary sequence, except that particular coils may have lower RF ring-down time that can contribute to lower TE.

ImRiD can be used as a gold standard for MR image reconstruction procedures using machine learning. The number of training examples that can be obtained from this dataset can be infinite due to the nature of slicing arbitrary 2D slice from 3D space. In parallel, exemplary experiments can be performed in line with tests determined by the phantom makers such as those by ACR phantom and/or ADNI phantom. These tests can cover different aspects of MR image quality such as low contrast detectability, resolution, slice thickness, etc. This can be extended to other system phantoms such as the ISMRM NIST. (See, e.g., Reference 18). This can facilitate benchmarking of the reconstructions performed using deep learning in line with these prescribed tests by the phantom makers/approvers.

### Exemplary Deep Learning Reconstruction

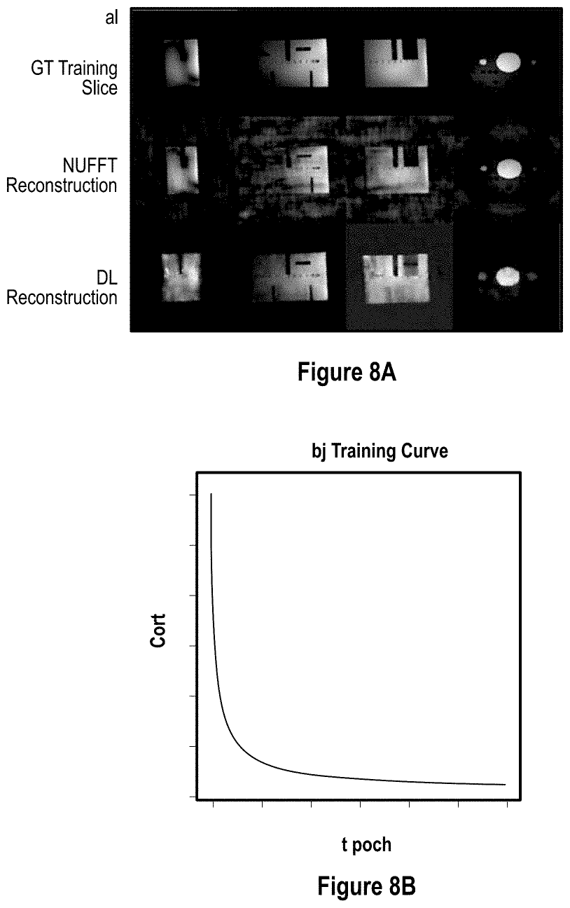

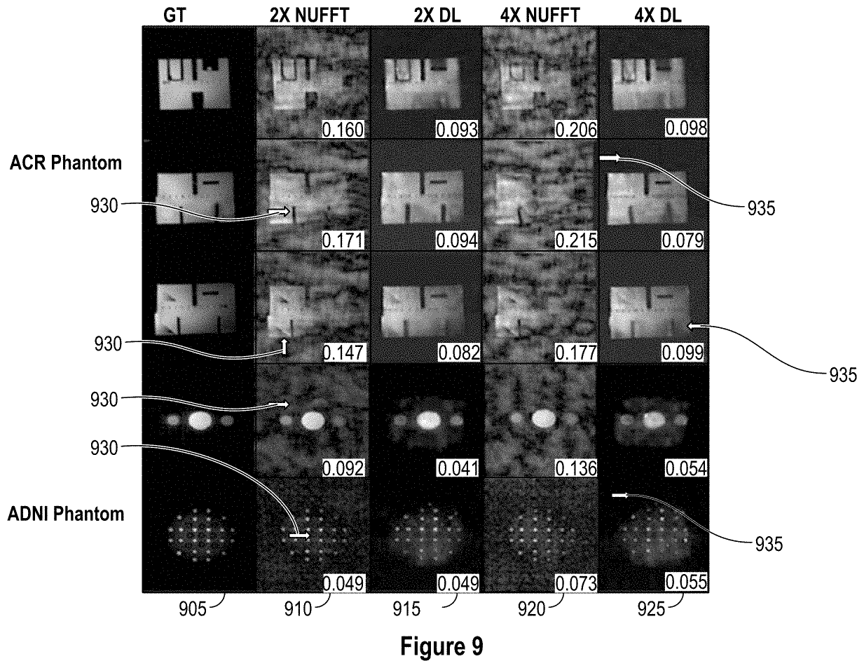

ImRiD was the exemplary dataset utilized for training the exemplary deep learning model. An exemplary advantage of this dataset can be that it docs not contain any anatomy specific shapes. ImRiD may only contain the mathematical transform between subsampled k-space and image. The exemplary U-net can train on complex data transforming k-space to images. FIG. 8A shows exemplary slice reconstruction results of the exemplary deep learning model compared with the ground truth and radial k-space reconstruction. The NUFFT results indicated a particular type of global noise spread evenly on the reconstructed images. The deep learning reconstruction suppressed that kind of noise. FIG. 8B shows an exemplary training curve of the cost versus epoch associated with the slice reconstruction results of FIG. 8A. The use of 300 epochs can bring the error from about 600 to about 50. FIG. 9 shows a set of exemplary image reconstructions of accelerated radial imaging according to an exemplary embodiment of the present disclosure. In particular, FIG. 9 illustrates a channel-wise deep learning reconstruction of accelerated radial imaging, which reconstructed under sampled data from another trajectory that was not employed in training. Column 905 shows the ground truth of ACR phantom and ADNI phantom. Column 910 illustrates the reconstruction image of 2× subsampled k-space. Column 915 shows the deep learning reconstruction of 2× subsample k-space. Column 920 illustrates the reconstruction image of 4× subsampled k-space. Column 925 shows the exemplary deep learning reconstruction of images. The background noise due to the subsampling was removed. Arrows 930 indicate where the traditional radial NUFFT performs better and arrows indicate 935 indicate where the exemplary deep learning reconstruction performs better. The exemplary RMSE error compared to the ground truth is shown on the bottom right of each image.

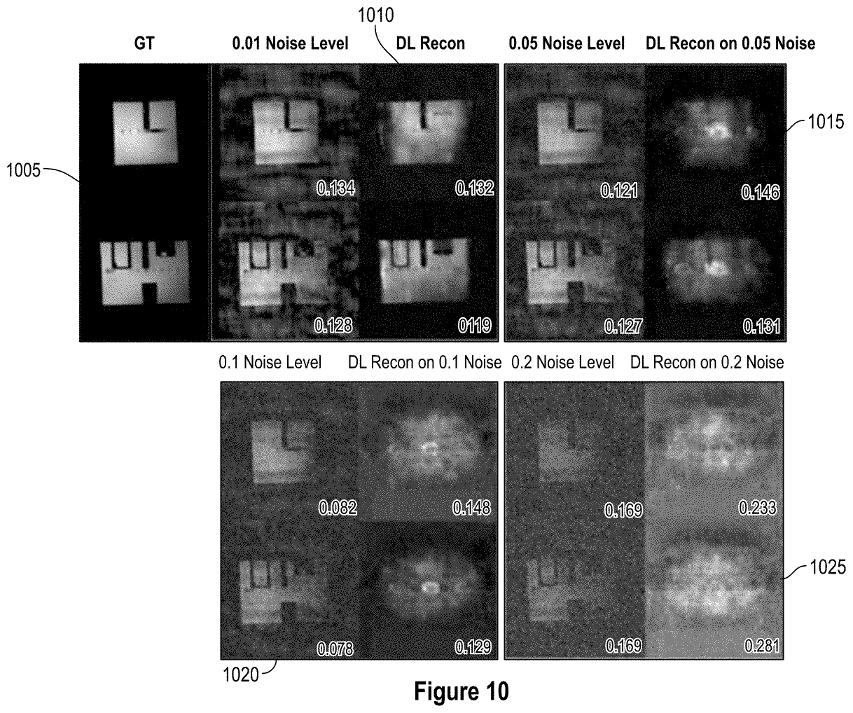

FIG. 10 shows a set of images having different noise levels according to an exemplary embodiment of the present disclosure. In particular, FIG. 10 shows channel-wise deep learning reconstruction of images when adding different level of noise. GT image 1005 was first non-uniform Fourier transformed to radial k-space. Then, the inverse NUFFT was performed to obtain the radial reconstruction of the image. Different noise levels were added to the radial recon image, which resulted in image 1010 having a 0.01 noise level, image 1015 having a 0.05 noise level, image 1020 having a 0.1 noise level, and image 1025 having a 0.2 noise level. Images 1010-1025 were Fourier Transformed to k-space and normalized to the input to test the network. The RMSE error compared to the ground truth is shown on the bottom right of each image.

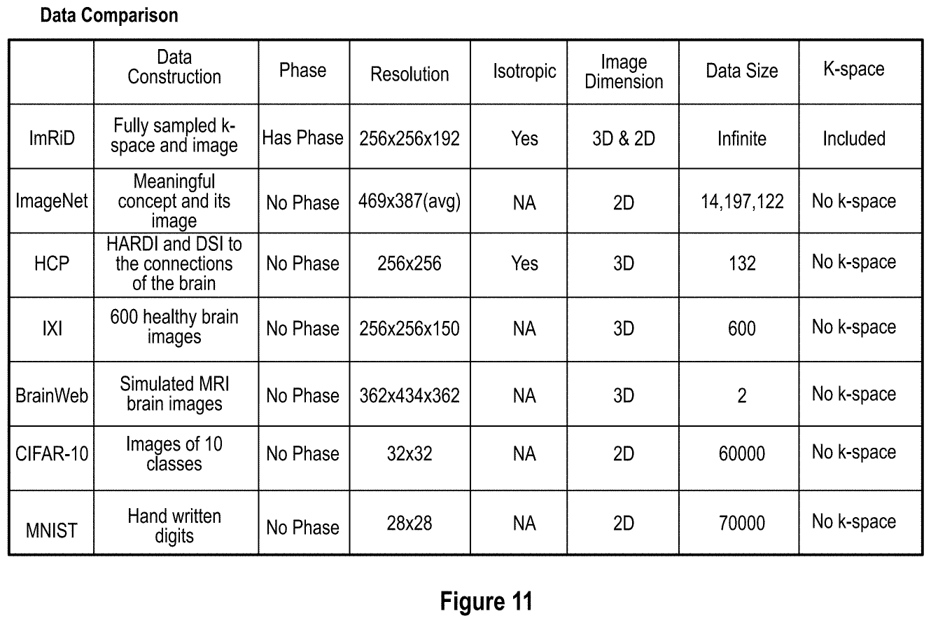

FIG. 11 is an exemplary table comparing various datasets according to an exemplary embodiment of the present disclosure. In particular. FIG. 11 illustrates different data sets available for exemplary machine learning procedures for image reconstruction and analysis. The exemplary database can include k-space data. 2D/3D information, as well as options to slice the image into multiple smaller image volumes or slices.

The body coil switching times dictate the UTE that was achieved. The exemplary system, method and computer-accessible medium, according to an exemplary embodiment of the present disclosure, can be flexible to accommodate other hardware specification as well. The exemplary system, method and computer-accessible medium was not performed on a knee TR coil which can enhance signal-to-noise ratio; however coil selection may not impact the exemplary sequence. The 0.2 ms TE was achieved with Pulseq. There can be some artifacts caused by the space between the subject and the coil since a body coil was used. A particular knee coil that can be closer to the subject can reduce the artifact. Pulseq can generate a 2D or 3D sequence. The 2D sequence can be in line with deep learning reconstruction procedures that become a close-loop architecture for rapid prototyping front acquisition to reconstruction.

As compared to other deep learning reconstruction methods, the exemplary method and system according to the exemplary embodiments of the present disclosure, can provide an improved memory efficiency in a high resolution. The exemplary U-net architecture may not utilize fully connected layers, which can utilize less memory and can be easier to train as compared with fully connected layers. The exemplary image reconstruction network can learn the mathematical transform on the anatomy specific shape. The exemplary deep learning based reconstruction method also performs better when the current task only has limited information or a relatively high amount of noise.

Corresponding sequences can be designed in Pulseq that can generate a radial trajectory and sequence for single slice GRE. The sequence can be applied to the scanner from different vendors, including Siemens, GE, Broker, and the exemplary deep learning neural network can be used to perform the reconstruction. The exemplary model was trained purely based on an ImRiD dataset, which can contain only the mathematical transform and can exclude the anatomy specific shape.

As compared to other datasets such as ImageNet (see, e.g., Reference 19), IXI dataset (see. e.g., Reference 20) and BrainWeb (see. e.g., Reference 21). ImRiD may not be image-oriented, but raw-oriented, indicating that the k-space of the raw data can be preserved. By preserving the k space, the database can preserve the phase information in the frequency domain that can typically be missed in image-only databases. Other parameters including isotropic voxel size. high resolution, can all be optimized for the purpose of image reconstruction. The exemplary data set can be utilized as a standard training data set for deep learning MR image reconstruction procedures for the following reasons:
(1) MR data from these phantoms are typically employed to test/calibrate the system as well as protocols;
(2) The complex image data captures the phase, noise and related characteristics of the system;
(3) Image processing procedures to slice an acquired 3D complex volume with high resolution can provide an infinite number of slices and therefore the unrestricted size of examples to train on;
(4) Extension to include acquisition methods lied to hardware such as parallel imaging, selective excitation can be incorporated;
(5) This library could be then also used lo under-sample k-space with different non-Cartesian trajectories to perform transform learning of under-sampled data; and
(6) The ground truth/construction of the phantom can be well specified and purposely designed.

### Exemplary Conclusion

The Pulseq and GPI combination of sequence design and image reconstruction can provide a powerful system and method for both developers and researchers who are working on MR imaging sequence design to create new sequences. Pulseq has the property of high-level programming while not sacrificing precise control of variables and time. It can maintain the degree of freedom for the designer in terms of varying the methods while simplifying the process of coding and transferring between different vendors' machine. The GPI is a powerful graphical programming tool that can reconstruct images efficiently, with a clear and precise visualization of the data flow. The UTE sequence can be produced, and the data from the scanner can be reconstructed. The Pulseq framework may have no restrictions to either the design of the sequence or the performance of the scanner.

The number of naming examples that can be obtained from this dataset can be infinite due to the nature of slicing 2D planes out of a 3D volume. In parallel, researchers can perform the experiment detailed in this work readily, easily and in line with tests determined by respective guidelines such as those provided by ACR and/or ADNI. These tests can cover different aspects of MR image quality, such as low contrast detectability, resolution, slice thickness, slice accuracy, etc. This can be extended to other system phantoms such as the ISMRM NIST. This property can facilitate benchmarking the reconstructions performed using deep learning in line with these prescribed tests by the phantom makers/approvers. The exemplary system, method, and computer-accessible medium, according 10 an exemplary embodiment of the present disclosure, can be beneficial for researchers who utilize data to train MR image reconstruction models since reconstruction procedures trained based on these phantoms can eater to multiple anatomies and related artifacts. Therefore, the exemplary model can be trained to learn the transform rather than be restricted by the anatomy.

The exemplary U-net can be used for a particular amount of data to train the network. For example, the U-net was able to suppress a lot of background noise due to the radial reconstruction. It illustrated superior performance when reconstructing two times and four times radial subsample k-space.

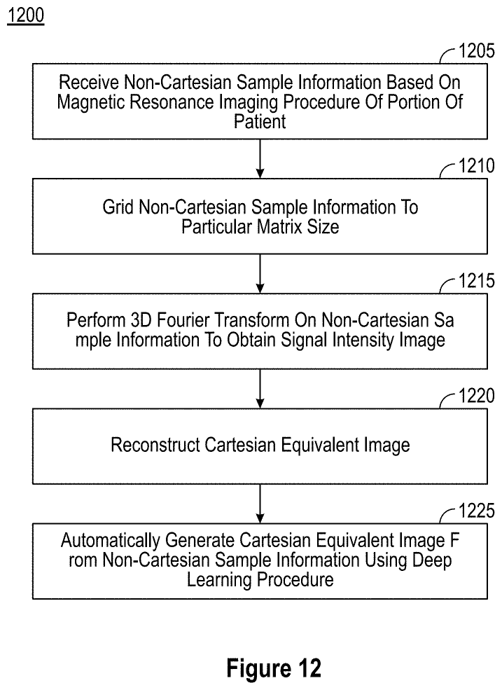

FIG. 12 shows a flow diagram of an exemplary method 1200 for generating a Cartesian equivalent image of a patient according to an exemplary embodiment of the present disclosure. For example, at procedure 1205, non-Cartesian sample information based on a magnetic resonance imaging (MRI) procedure of a portion of the patient can be received. At procedure 1210, the non-Cartesian sample information can be gridded to a particular matrix. At procedure 1215, a 3D Fourier transform can be performed on the non-Cartesian sample information to obtain a signal intensity image size. At procedure 1220, the Cartesian equivalent image can be reconstructed. At procedure 1225, the Cartesian equivalent image can be automatically generated using a deep learning procedure.

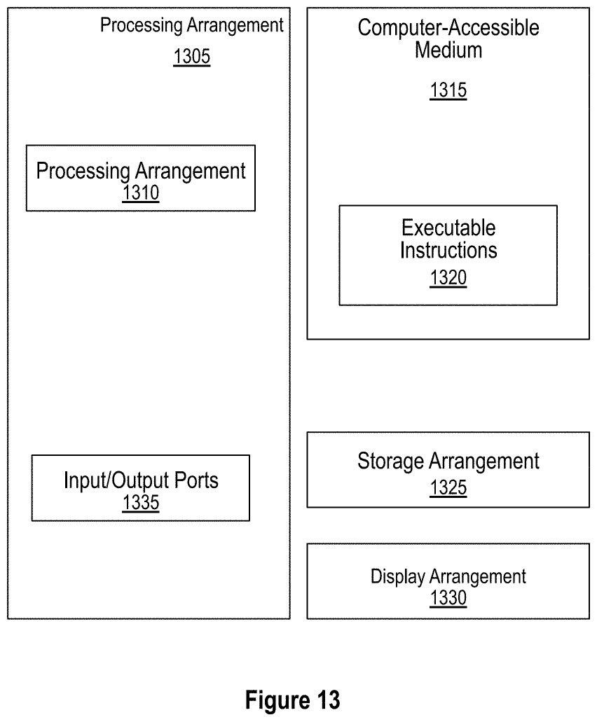

FIG. 13 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1305. Such processing/computing arrangement 1305 can be, for example entirely or a part of, or include, but not limited to, a computer/processor 1310 that can include, for example one or more microprocessors, and use instructions stored on a computer-accessible medium (e.g., RAM. ROM, hard drive, or other storage device).

As shown in FIG. 13, for example a computer-accessible medium 1315 (e.g., as described herein above, a storage device such as a hard disk, floppy disk, memory stick, CD-ROM, RAM, ROM, etc., or a collection thereof) can be provided (e.g., in communication with the processing arrangement 1305). The computer-accessible medium 1315 can contain executable instructions 1320 thereon. In addition or alternatively, a storage arrangement 1325 can be provided separately from the computer-accessible medium 1315, which can provide the instructions to the processing arrangement 1305 so as to configure the processing arrangement to execute certain exemplary procedures, processes, and methods, as described herein above, for example.

Further, the exemplary processing arrangement 1305 can be provided with or include an input/output pons 1335, which can include, for example a wired network, a wireless network, the internet, an intranet, a data collection probe, a sensor, etc. As shown in FIG. 13, the exemplary processing arrangement 1305 can be in communication with an exemplary display arrangement 1330, which, according to certain exemplary embodiments of the present disclosure, can be a touch-screen configured for inputting information to the processing arrangement in addition to outputting information from the processing arrangement, for example. Further, the exemplary display arrangement 1330 and/or a storage arrangement 1325 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.
[1] Layton, Kelvin J., et al. “Pulseq: A rapid and hardware-independent pulse sequence prototyping framework.” Magnetic resonance in medicine 77.4 (2017): 1544-1552.
[2] Golkov, Vladimir, et al. “Q-space deep learning: twelve-fold shorter and model-free diffusion MRI scans.” IEEE transactions on medical imaging 35.5 (2016): 1344-1351.
[3] Wang, Ge. et al. “Image reconstruction is a new frontier of machine learning.” IEEE transactions on medical imaging 37.6 (2018): 1289-1296.
[4] Işin, Ali, Cem Direkoğlu, and Melike Şah. “Review of MRI-based brain tumor image segmentation using deep learning methods.” Procedia Computer Science 102 (2016): 317-324.
[5] Liu. Siqi, cl al. “Early diagnosis of Alzheimer's disease with deep learning.” Biomedical Imaging (ISBI), 2014 IEEE 11th International Symposium on. IEEE, 2014.
[6] Ronneberger, Olaf, Philipp Fischer, and Thomas Brox. “U-net: Convolutional networks for biomedical image segmentation.” International Conference on Medical image computing and computer-assisted intervention. Springer. Cham, 2015.
[7] Zhu, Bo, et al. “Image reconstruction by domain-transform manifold learning.” Nature 555.7697 (2018): 487, on pre-processing before Fourier transform(8) or post-processing after the Fourier transform(9)
[8] Yoseob Han. Jong Chul Ye, et al. “Non-cartesian k-space deep learning for accelerated MRI” ISMRM machine learning workshop (20l8)
[9] Hyun, Chang Min, et al. “Deep learning for undersampled MRI reconstruction.” Physics in medicine and biology (2018).
[10] Togao, Osamu, et al. “Ultrashort echo time (UTE) MRI of the lung: assessment of tissue density in the lung parenchyma.” Magnetic resonance in medicine 64.5 (2010): 1491-1498.
[11] Mugler III, John P., and James R. Brookeman. “Three-dimensional magnetization-prepared rapid gradient-echo imaging (3D MP RAGE).” Magnetic Resonance in Medicine 15.1 (1990): 152-157.
[12] Chen. Chien-Chuan. et al. “Quality assurance of clinical MRI scanners using ACR MRI phantom: preliminary results.” Journal of digital imaging 17.4 (2004): 279-284.
[13] Gunter, Jeffrey L., et al. “Measurement of MRI scanner performance with the ADNI phantom.” Medical physics36.6Part1 (2009): 2193-2205.
[14] https://github.com/imr-framcwork/imr-framcwork/tree/mastcr/Matlab/Recontruction/ImRiD
[15] Yu. Daniel F., and Jeffrey A. Fessler. “Edge-preserving tomographic reconstruction with nonlocal regularization.” IEEE transactions on medical imaging 21.2 (2002): 159-173.
[16] https://drive.google.com/drivc/foldcrs/li7C2bK7psdc/z91a2BZVd3RyopXxVC8zj?usp-sharing
[17] Keenan, Kathryn E., et al. “Comparison of T1 measurement using ISMRM/NIST system phantom.” ISMRM 24th Annual Meeting. No. Program #3290. 2016.
[18] Krizhevsky, Alex. Ilya Sutskever, and Geoffrey E. Hinton. “Imagenet classification with deep convolutional neural networks.” Advances in neural information processing systems, 2012.
[19] Wu. Guorong, et al. “Unsupervised deep feature learning for deformable registration of MR brain images.” International Conference on Medical Image Computing and Computer-Assisted Intervention. Springer, Berlin, Heidelberg, 2013.
[20] Varela, Francisco, et al. “The brainweb: phase synchronization and large-scale integration.” Nature reviews neuroscience 2.4 (2001): 229.

1. A non-transitory computer-accessible medium having stored thereon computer-executable instructions for generating at least one Cartesian equivalent image of at least one portion of at least one patient, wherein, when a computer arrangement executes the instructions, the computer arrangement is configured to perform procedures comprising:
receiving non-Cartesian sample information based on a magnetic resonance imaging (MRI) procedure of the at least one portion of the at least one patient; and
automatically generating the at least one Cartesian equivalent image from the non-Cartesian sample information using at least one deep learning procedure.

2. The computer-accessible medium of claim 1, wherein the non-Cartesian sample information is Fourier domain information.

3. The computer-accessible medium of claim 1, wherein the non-Cartesian sample information is undersampled non-Cartesian sample information.

4. The computer-accessible medium of claim 1, wherein the MRI procedure includes an ultra-short echo time (UTE) pulse sequence.

5. The computer-accessible medium of claim 4, wherein the UTE pulse sequence includes at least one delay and a spoiling gradient.

6. The computer-accessible medium of claim 1, wherein the computer arrangement is configured to automatically generate the at least one Cartesian equivalent image by reconstructing the at least one Cartesian equivalent image.

7. The computer-accessible medium of claim 6, wherein the computer arrangement is configured to reconstruct the at least one Cartesian equivalent image using a sampling density compensation with a tapering of over a particular percentage of a radius of a k-space.

8. The computer-accessible medium of claim 7, wherein the particular percentage is about 50%.

9. The computer-accessible medium of claim 7, wherein the computer arrangement is configured to reconstruct the at least one Cartesian equivalent image by gridding the non-Cartesian sample information to a particular matrix size.

10. The computer-accessible medium of claim 9, wherein the computer arrangement is configured to reconstruct the at least one Cartesian equivalent image by performing a 3D Fourier transform on the non-Cartesian sample information to obtain at least one signal intensity image.

11. The computer-accessible medium of claim 1, wherein the at least one deep learning procedure includes at least 20 layers.

12. The computer-accessible medium of claim 11, wherein the at least one deep learning procedure includes convolving an input at least twice.

13. The computer-accessible medium of claim 12, wherein the at least one deep learning procedure includes max pooling the second layer.

14. The computer-accessible medium of claim 1, wherein the at least one deep learning procedure includes at least one of convolving or max pooling a first 10 layers.

15. The computer-accessible medium of claim 1, wherein the at least one deep learning procedure includes forming a 13^{th} layer by concatenating a 9^{th} layer with a 12^{th} layer.

16. The computer-accessible medium of claim 1, wherein the at least one deep learning procedure includes convolving a last 4 layers.

17. The computer-accessible medium of claim 1, wherein the at least one deep learning procedure includes maintaining a particular resolution from layer 13 to layer 18.

18. The computer-accessible medium of claim 1, wherein the at least one deep learning procedure includes 13 convolutions, 4 deconvolutions, and 4 combinations of maxpooling and convolution.

19. A method for generating at least one Cartesian equivalent image of at least one portion of at least one patient, comprising:
receiving non-Cartesian sample information based on a magnetic resonance imaging (MRI) procedure of the at least one portion of the at least one patient; and
using a computer hardware arrangement, automatically generating the at least one Cartesian equivalent image from the non-Cartesian sample information using at least one deep learning procedure.

20-36. (canceled)

37. A system for generating at least one Cartesian equivalent image of at least one portion of at least one patient comprising:
a computer hardware arrangement configured to:
receive non-Cartesian sample information based on a magnetic resonance imaging (MRI) procedure of the at least one portion of the at least one patient; and
automatically generate the at least one Cartesian equivalent image from the non-Cartesian sample information using at least one deep learning procedure.

38-54. (canceled)
